(19) United States
(12) Patent Application Publication
(10) Pub. No.: US 2021/0172923 A1
(43) Pub. Date: Jun. 10, 2021

(54) DEVICES, COMPOSITIONS AND METHODS FOR USE IN DETECTING CONTAMINATING HEAVY METALS IN WATER SOURCES

(71) Applicant: Spout, Denver, CO (US)

(72) Inventors: Ari Kaufman, Denver, CO (US); Robert A. Masinter, Arvada, CO (US); Diane A. Blake, New Orleans, LA (US); Joseph Kaeller, Temecula, CA (US); Bart J. Massee, Kirkland, WA (US); Brian Heinze, Ft. Collins, CO (US)

(21) Appl. No.: 17/247,247

(22) Filed: Dec. 4, 2020

Related U.S. Application Data
(60) Provisional application No. 62/944,739, filed on Dec. 6, 2019.

(51) Int. Cl. G01N 33/18; G01N 21/78; B01L 3/00; C07K 16/44

(52) U.S. Cl. CPC G01N 33/1813; G01N 21/78; G01N 2021/7759; C07K 16/44; B01L 3/508

(57) ABSTRACT

The disclosure relates to electronic reader devices, detection compositions and methods for detecting heavy metals (e.g., lead) in water samples using a simple, rapid and highly selective immunoassay system, including lateral and upward flow assay systems. The immunoassay is designed to detect very low levels of a contaminating analyte such as lead at less than 1 ppb with high specificity and selectivity from any common water source (e.g., tap or drinking water). Advantageously, implementation of the assay requires no formal technical training or expensive equipment or reagents and is provided with a multi-use reader that can be used for 50 samples or more. The immunoassay sample collector systems are consumable (single use) and can be purchased in any desired quantity. Assay results are wirelessly transmitted from the reader using a SafeSpout-designed app., which provides important information based on the test results.

### CROSS-REFERENCE TO RELATED APPLICATIONS

This application claims the benefit of priority from U.S. Provisional Application Ser. No. 62/944,739, filed on Dec. 6, 2019, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 3, 2020, is named 052652-501001US_SL_ST25.txt and is 4,073 bytes in size.

### FIELD OF THE DISCLOSURE

The subject matter described herein generally relates to sample collection and preparation systems, contaminant detection methods and novel reagents, sample reader instruments, related software and smartphone applications, for use in detecting contaminating heavy metals (e.g., lead) in drinking water (e.g., potable water sources) and in environmental and industrial water (e.g., non-potable sources) using lateral or upward flow assay technology. Specifically, the disclosure relates to immunosensing devices for simple implementation and detection of total lead in drinking water, tap water, and other readily consumable types of water sources as well as environmental and industrial water sources. Advantageously, the disclosure provides devices, compositions, and methods for detecting both particulate and dissolved lead that are simple to use, highly sensitive, highly specific, and provide rapid electronic readouts without the use of sophisticated equipment, multiple user steps, and specialized know-how normally required for the detection of heavy metals.

### BACKGROUND OF THE DISCLOSURE

Lead contamination is a serious worldwide health and environmental issue. Lead can bioaccumulate in the body and have serious long-term health consequences such as kidney problems and high blood pressure in adults and physical and mental development delays in children (Jones, A. 2009). The US-EPA has set a National Primary Drinking Water Regulations (NPDWR) goal of zero lead in municipal drinking water systems, but an action level of 15 ppb in all municipal water systems. A recent study revealed that up to 75% of the total mass of lead measured in tap water comes from the lead service lines that connect residences with water mains (AWWA Research Foundation, 2008). It has been reported that up to 30% of total lead measured in U.S. municipal drinking water occurs as particulates (>45 μm) derived from corrosion of lead service lines or older premises plumbing lines (Deshommes, E. et al., 2010). Further, lead in drinking water is often present in colloidal form (<45 μm in size) (Deshommes, E. et al., 2010), which increases the fraction of total lead in drinking water that is not in the dissolved, ionic state. Currently available technology using analytical test strips only provides qualitative measures of dissolved, ionic lead and as a result underestimates total lead load in drinking water samples. As citizens become increasingly aware of the dangers associated with the ingestion and inhalation of lead, it is becoming increasingly desirable to provide simple, handheld devices that can rapidly, accurately and quantitatively measure the amount of total (particulate and dissolved) lead in municipal and other commonly used water sources. Ideally, such systems should be user friendly and have a minimal number of procedural steps while yielding reliable and reproducible results, even when performed by untrained individuals.

U.S. Pat. No. 5,019,516 describes a method of extracting lead from a sample of drinking water followed by quantitative determination of the total lead in the sample. The process described in U.S. Pat. No. 5,019,516 has been developed into the LeadTrak™ system (Hach) and involves extraction, complexation, neutralization and colorimetric determination of lead in a water sample. The system has been adapted for use with a hand-held pocket colorimeter but sample preparation and accuracy of results requires skilled users, costly reagents and is time-consuming.

U.S. Pat. No. 5,089,663 describes new rigid chelating structures, as well as methods of preparing them and using them in preparing radiometal labeled immunoconjugates. The new chelates include cyclohexyl EDTA, the trans forms of cyclohexyl DTPA and TTHA, and derivatives thereof.

U.S. Pat. No. 8,859,265 describes a lateral flow immunoassay device for qualitative and quantitative analysis of an analyte in whole blood with improved accuracy.

U.S. Pat. No. 8,614,101 discloses lateral flow assay devices and methods that incorporate lysis agents for use in a point-of-care testing device. Cell samples (e.g., red blood cells, leukocytes) containing a suspected analyte of interest (e.g., drugs, viruses, nucleic acids, RNA, etc.) are loaded in a sample application area comprising a paper strip and flow until they encounter a pre-loaded lysis agent where they migrate through to a conjugate zone and a detection zone. Immunoassays are described for use in detecting analytes after cell lysis.

U.S. Pat. No. 6,699,722 discloses methods and devices for the qualitative and quantitative detection of an analyte in a sample. The methods describe a positive detection assay system, i.e., that a stronger signal corresponds to more analyte present. Devices of the disclosed subject matter may include a sample application area, a mobilization zone including a mobile analyte analog, primary and secondary capture areas each of which includes an immobilized binding partner having a binding affinity for the analyte being tested for detection of the analyte analog. A tracer conjugate is used in the mobilization zone that migrates slightly behind the analyte sample so any sample contacts the binding partner before the conjugate. The results are visualized based on either the presence and/or intensity of the detectable signal provided by the conjugate that binds in the secondary capture area.

U.S. Pat. No. 7,109,942 discloses a test device for determination of an analyte in a liquid sample, comprising: (a) a nitrocellulose carrier, (b) a binding reagent effective to capture analyte, when present, in a defined detection zone of the nitrocellulose carrier; (c) a labeled reagent which is freely mobile in the nitrocellulose carrier in the presence of the liquid sample, said labeled reagent being selected such that it is captured in the detection zone when analyte is present in the liquid sample; (d) a sample receiving member; and (e) a control zone, disposed on or in the nitrocellulose carrier on a side of the detection zone remote from the sample receiving member. The control zone comprises a control-binding reagent, which binds the labeled reagent whether or not analyte is present in the sample. Liquid sample applied to the sample receiving member is transported to and then along the length of the nitrocellulose carrier to pass through the detection zone, and the detection of labeled reagent in the detection zone is indicative of the presence of analyte in the liquid sample.

U.S. Pat. No. 6,020,147 discloses a device for detecting the presence of an analyte in a carrier liquid suspected of containing the analyte. The device comprises a liquid permeable solid medium which defines a path for fluid flow capable of supporting capillary flow, along which are i) a site for application of the carrier liquid, ii) a diffusively bound labeled reactant specific for the analyte or a chemical moiety which is itself the reaction product of the analyte with another chemical moiety, said labeled reactant being capable of flowing along the flow path, wherein said diffusively bound labeled reactant and said analyte or chemical moiety are of a specific ligand-receptor (antigen-antibody) pair, and iii) one or more zones spaced along the flow path, each zone having a predetermined amount of a reactant bound to it which is specific for either the analyte or a chemical moiety which is itself the reaction product of the analyte with another chemical moiety. The device can be used by contacting a carrier liquid with the application site in such a manner that permits the liquid to pass along the flow path by capillary flow such that analyte or reaction product of the analyte with another chemical moiety becomes bound to both the labeled reactant and the reactant bound to the solid medium. The labeled reactant, with the reactant bound to the solid medium, sandwiches the analyte or a chemical moiety, which is itself the reaction product of the analyte with another chemical moiety.

U.S. Pat. No. 6,352,862 discloses analytical devices that are suitable for use in the home, clinic or doctor's surgery and which are intended to give an analytical result rapidly and which require the minimum degree of skill and involvement from the user. The use of test devices in the home to test for pregnancy and fertile period (ovulation) is now commonplace.

U.S. Pat. No. 6,001,658 describes a test strip device that can be used by itself or with an associated housing assembly with a diffusible, labeled binding partner that binds with analyte, an immobilized analyte, and a detection area containing an immobilized antibody. The test strip assay provides a semi-quantitative reading of the analyte concentration.

U.S. Pat. No. 5,622,871 discloses an analytical test device useful for example in pregnancy testing, includes a hollow casing constructed of moisture-impervious solid material, such as plastics materials, containing a dry porous carrier which communicates indirectly with the exterior of the casing via a bibulous sample receiving member which protrudes from the casing such that a liquid test sample can be applied to the receiving member and permeate therefrom to the porous carrier, the carrier containing in a first zone a labelled specific binding reagent is freely mobile within the porous carrier when in the moist state, and in a second zone spatially distinct from the first zone unlabeled specific binding reagent for the same analyte which unlabeled reagent is permanently immobilized on the carrier material and is therefore not mobile in the moist state, the two zones being arranged such that liquid sample applied to the porous carrier can permeate via the first zone into the second zone, and the device incorporating an aperture in the casing, enabling the extent (if any) to which the labelled reagent becomes bound in the second zone to be observed. The device may include a removable cap for the protruding bibulous member.

U.S. Pat. No. 5,451,507 describes a two-zone, disconnected immunographic method where the first zone has non-diffusively bound reagent that binds with the component, e.g., an analyte complex or conjugate, bound to, or capable of being bound to, a member of a signal-producing system. When the analyte to be tested is present it becomes bound upon entry into the second zone. The concentration of analyte is directly determined on the distance the component migrates into the second zone.

Marzo et al. (2013), Anal. Chem. 85:3532-3538 describe an integrated, lateral flow, paper-based immunodetection device for the detection of cadmium (Cd²⁺) in drinking water. The detection system is based on a competitive reaction between a Cd-EDTA-BSA-gold nanoparticle conjugate deposited on the conjugation pad strip and the Cd-EDTA complex formed in the analysis sample for the same binding sites of a monoclonal antibody specific to Cd-EDTA but not free Cd²⁺, which is immobilized onto a detection test line. Cd²⁺ detection levels were as low as 0.1 ng/ml and quantitation levels as low as 0.4 ng/ml.

Kuang et al. (2013), Readers 13: 4214-4224 describe a rapid and sensitive lateral flow assay strip for the immunodetection of lead in drinking water. The system is based on detection of an analyte which migrates by capillary flow through a series of areas on a strip where the analyte is successively bound first to a chelator or conjugate and then to a specific antibody which recognizes the analyte-chelator of the complex or conjugate.

Khosraviani et al. (2000), Bioconjugate Chem. 11:267-277 disclose the generation and characterization of a monoclonal antibody (2C12) that recognizes a Pb(II)-cyclohexyldiethyenetriamine pentaacetic acid (CHXDTPA) complex. When CHXDTPA was used as the chelator in a complex or in a conjugate with BSA the addition of Pb(II) increased the affinity of the antibody for the complex more than 200-fold. Sensitivity of prototype immunoassays using Pb(II) could be modulated by changing the structure of the immobilized metal-chelate complexes and/or the soluble chelator used to complex Pb(II) in the test solution.

D. Rahmi et al. (2007), Talanta 72:600-606; Y. Zhu et al. (2005), Bull Chem Soc Japan 78:107-115; and Y. Zhu et al. (2004), Bull Chem Soc Japan 77:1834-1842, describe a method for removing dissolved salts and Ca²⁺ in seawater samples that can be adapted for the sample preparation method necessary to treat tap water.

Thus, there remains a need in the art for a rapid, highly sensitive, and user-friendly assay device to detect very low levels of particulate, colloidal and dissolved lead, i.e., total lead, in municipal and household drinking/tap water systems and other water sources. The ideal assay should involve a minimal number of procedural steps, yield reliable and reproducible results, and be simple for untrained individuals for use at home, in the field and in work environments.

### SUMMARY OF THE DISCLOSURE

The disclosure provides reader devices, detection compositions and detection methods for identifying and quantifying analytes in drinking water at the ng/ml (ppb) level. Embodiments of the disclosure provide an assay device, for example, a lateral flow or chromatographic assay, that reliably detects and accurately measures dissolved (Pb(II)) and particulate lead in drinking water at low concentrations (<1 ppb) even in the presence of potentially interfering analytes. In some embodiments, a sample pre-treatment system prevents the assay from being subject to interference from calcium and other metals and chemicals commonly present in drinking water. In some embodiments, a sample pre-treatment system solubilizes particulate and colloidal lead that may exist in drinking water as a result of corrosion from municipal pipes and plumbing systems. Results from the methods and devices disclosed herein may be quantitatively read using an opto-electronic reader such as a scanner.

Provided herein is a method of detecting and quantitating an analyte in a sample comprising the steps of: a) obtaining a sample potentially containing an analyte of interest; b) transferring the sample to a sample collector pre-treating the sample to prepare the sample for detection and quantitation of the analyte; c) contacting the pre-treated sample with a test strip; d) assaying the pre-treated sample using the test strip to determine the concentration of the analyte in the pre-treated sample; and e) quantitating the concentration of the analyte, wherein the concentration of the analyte is measured qualitatively and quantitatively using the test strip.

The disclosure further provides a method of detecting and quantitating an analyte in the sample comprising an immunoassay test strip, wherein a sample application area and a detection area are on a chromatographic immunoassay test strip, and the test strip further comprises a sample pad, wherein the pre-treated sample is applied to or first contacted with the test strip and a conjugate area or pad, wherein the conjugate pad comprises at least one labeled binding partner that is able to migrate with the sample medium, and a capture area or pad, wherein the capture pad comprises a test line comprising chelator-conjugates and a control line comprising anti-species antibodies, and an absorbant pad, wherein the sample flow terminates.

The methods and devices disclosed herein may be used to detect an analyte (e.g., lead) in various types of water sources in a liquid sample selected from the group consisting of tap water, well water, unfiltered drinking water, filtered drinking water, household plumbing water contained within pipes, bottled water, municipal water, aquifer water, wastewater including industrial wastewater sources, effluent and river water.

Any known source of potable or consumable water or non-potable or industrial water that might contain an analyte of interest (e.g., lead) can be easily detected using the devices, compositions and methods of the present disclosure. In some embodiments, the method is disclosed wherein the sample is a liquid sample selected from the group consisting of tap water, well water, unfiltered drinking water, filtered drinking water, household plumbing water contained within pipes, bottled water, municipal water, aquifer water, wastewater including industrial wastewater sources, effluent and river water.

In some embodiments, the analyte comprises a heavy metal. In some embodiments, the heavy metal is selected from lead Pb(II), chromium Cr(II), arsenic Ar(II), cadmium Cd(II), and mercury Hg(II). In some embodiments, the heavy metal is lead Pb(II).

In some embodiments, the assay includes a multi-use reader device, single-use sample collection and preparation systems, detection compositions and detection methods that provide for quantitative assessments of the presence of an analyte (e.g., lead) in a water sample, for example, by colorimetric or fluorescence analysis. In some embodiments, the reader device, sample collection and preparation system, detection compositions and detection methods are provided for quantitative determination of the presence of total lead in a water sample. In some embodiments, an assay system is provided that consists of one or more lateral- or upward-flow immunoassay strips enclosed within a plastic housing with an internal design to manage fluidics, including a top housing and a bottom housing, a sample handling component (e.g., a collection and pre-treatment system), an opto-electronics assembly for sensing, detection analysis, power and electronic communications, and a mechanical assembly for actuation of the system elements. In an aspect of the disclosure, the analyte reader device is adapted to communicate with a software application to allow rapid, quantitative interpretation of analyte concentrations in a water sample. In some embodiments, the assay may be incorporated into a disposable assay cassette, which is then integrated with a sample collection and preparation system, collectively the single-use consumable. The multi-use reader may include illumination, a motor drive, Bluetooth transmission (BLE) capability, integrated circuits and micro-controllers, programmable firmware, and batteries. In some embodiments, software is provided consisting of a smartphone application (“app”) and a back-end software platform for communicating analyte concentration results from the reader device to the user.

The assay system of some embodiments of the present disclosure may also provide an immunoassay strip comprised of chelator (e.g., CHXADTPA), chelator-conjugates (CHXADTPA-conjugate), a recombinant antibody, reagents, signaling nanoparticles, sample application pads, capture pads, and absorbent pads. In some embodiments, the immunoassay strip may be comprised of different sections including a sample application area or pad, a conjugate pad, a capture pad, which includes a test line and a control line and an absorbent pad. The conjugate pad may include different reagents that bind the lead in the sample. For example, the conjugate pad may contain a chelator such as CHXADTPA that binds lead to form Pb(II)-CHXADTPA complexes. The conjugate pad may also contain chelator conjugates, for example CHXADTPA conjugated to bovine serum albumin (BSA), which forms in the presence of Pb(II), Pb(II)-CHXADTPA-BSA conjugates. The capture pad test line includes monoclonal antibodies specific for bound lead contained in either Pb(II)-CHXADTPA complexes or Pb(II)-CHXADTPA conjugates. Monoclonal antibodies are covalently linked to gold nanoparticles (AuNP) for colorimetric detection and the monoclonal antibodies-AuNP complexes are embedded at the test line. In some embodiments, particularly if a lateral flow assay is employed, the sample receiving pad, conjugate pad, capture pad, test and control lines and absorbent pad are in continuous fluid contact with each other. In alternative embodiments, where a sample pad or sample receiving area is not required, for example, when the chromatographic assay is employed and sample flows upward, the conjugate pad, capture pad, test and control lines and absorbent pad are in continuous fluid contact with each other.

In some embodiments, the recombinant antibody in the test line may have a higher affinity for the Pb(II)-CHXADTPA-conjugate than for the Pb(II)-CHXADTPA complex such that the Pb(II)-conjugate may be bound at the test line and the Pb(II)-CHXADTPA complex may pass through to the control line. In some embodiments, the monoclonal antibodies in the test line have an equal affinity for both the Pb(II)-CHXADTPA complex and the Pb(II)-CHXADTPA-conjugate. In some embodiments, the monoclonal antibodies embedded in the test line may have a higher affinity for the Pb(II)-CHXADTPA complexes and the Pb(II)-CHXADTPA complexes may bind at the test line.

Embodiments of the assay of the present disclosure may involve introducing a liquid water sample suspected to contain lead for testing onto a strip at the exit port or distribution chamber of the sample collection preparation device and permit the sample to migrate up or along the strip by capillary action from the base of the conjugate pad through the capture pad test and control lines and finally to the absorbent pad. The reporter group or detection molecule may be present in the sample receiving pad, the conjugation pad, in the path of migration but before the test and control lines or applied separately to the strip. In some embodiments, the detection molecule is covalently linked to the monoclonal antibody. In some embodiments, the Pb(II)-CHXADTPA-conjugate migrates behind the Pb(II)-CHXADTPA complex so that the Pb(II)-CHXADTPA complex reaches the test line ahead of the Pb(II)-CHXADTPA conjugate, thus reducing signal at the test line and binding to the control line with indirect antibody binding to anti-BSA antibodies.

In some embodiments, the heavy metal detection level in the pre-treated sample is at a concentration of between about <1 ppb to about 20 ppb. In some embodiments, the concentration of metal in the pre-treated sample is between <1 ppb and about 15 ppb, between <1 ppb and about 8 ppb, between <1 ppb and 5 ppb, between <1 ppb and 2 ppb. In some embodiments, the lead Pb(II) concentration in the pre-treated sample is <1 ppb.

The present disclosure further provides a system for detecting and quantitating an analyte in a sample comprising the steps of: a) obtaining a sample potentially containing an analyte of interest; b) transferring the sample to a sample collector for pre-treatment to prepare the sample for detection and quantitation of the analyte; c) contacting the pre-treated sample with a test strip; d) assaying the pre-treated sample using the test strip to determine the concentration of the analyte in the sample, wherein the concentration of the analyte is measured qualitatively and quantitatively using the test strip; e) qualitatively determining the concentration level of a metal detected in step d), wherein the result is determined within 60 minutes, within 45 minutes, within 30 minutes, within 20 minutes or within 15 minutes; and f) quantitatively determining the concentration level of a metal detected in step d), wherein the metal concentration level is recorded in a multi-use reader device. In some embodiments, the concentration data is wirelessly transferred to a smartphone app using a back-end software platform. In some embodiments, the sample collector in step b) and parts of the multi-use reader device in step f) are disposable or recyclable.

The present disclosure further provides a device for detecting and quantitating an analyte in a liquid sample comprising: an analyte detection reader 100; a sample collector 110; and an assay cassette 130, wherein the analyte detection reader is designed to hold the sample collector and the assay cassette for reading a sample. In embodiments, the sample collector further comprises a sample pre-treatment system 112. In some embodiments, the sample pre-treatment system 112 comprises: a water collection chamber 114; a blister ampoule containing acid 116; a distribution rod 118; an ion-exchange resin 120; a drain 121; a waste collection chamber 122; a separation chamber 123; sample splitting chambers 124; a blister or equivalent containing neutralizing reagent and chelator(s) 125; sample distribution chambers 126; and immunoassay strips 128a and 128b, wherein the immunoassay strips detect different concentration ranges of analyte.

In some embodiments, the immunoassay strips are for detecting and quantitating an analyte comprising: a base plate 202; a sample receiving pad 204a and a dried sample treatment pad 204b; a first conjugate pad area 206a and a second conjugate pad area 206b, wherein the first conjugate pad area and the second conjugate pad area are impregnated with a first binding partner bound to a detection reagent; and wherein the first binding partner bound to the detection reagent is able to flow along the immunoassay strips to an elongated analyte detection capture pad 208; a first capture area comprising a test line 210 immobilized with analyte chelator-conjugates, wherein the first binding partner bound to a detection reagent competes for binding sites at the test line; a second capture area comprising a control line 212 immobilized with a second binding partner; and an absorbent pad 214a and an absorbent reservoir pad 214b, wherein the absorbent pad acts as a wicking mechanism to regulate capillary flow from the sample receiving pad to the absorbent reservoir pad.

In some embodiments, the binding partner comprises a monoclonal antibody, a chimeric antibody, a CDR-grafted antibody, a single-chain (scFv) antibody, disulfide-linked Fvs (sdFv), Fab fragments, F(ab′) fragments, or antigen-binding fragments. In some embodiments, the monoclonal antibody is the monoclonal antibody designated 2C12, wherein 2C12 comprises light-chain and heavy-chain variable regions. In some embodiments, the light-chain and heavy-chain variable regions consist of the amino acid sequences of SEQ ID NO: 1 and SEQ ID NO: 2, respectively; wherein the complementarity-determining regions (CDR) in the light chain variable region of 2C12 consist of residues 24-39 (SEQ ID NO: 3), residues 55-61 (SEQ ID NO: 4) and residues 95-103 (SEQ ID NO: 5); and wherein the complementarity-determining regions (CDR) in the heavy-chain variable region of 2C12 consist of residues 26-35 (SEQ ID NO: 6), residues 50-65 (SEQ ID NO: 7) and residues 98-105 (SEQ ID NO: 8).

In some embodiments, the device further comprises chelators. In some embodiments, the chelators are selected from the group consisting of CHXDTPA, CHXEDTA, EDTA, EGTA, citrate, and ITCBE or the free acids of any of the foregoing. In some embodiments, the chelator is CHXDTPA.

In some embodiments, the device further comprises detection or labeling reagents. In some embodiments, the detection or labeling reagents are selected from the group consisting of gold nanoparticles (AuNP), latex microparticles, reporter groups including horseradish peroxidase (HRP) and alkaline phosphatase (AP), metal sol tags including silver, selenium and carbon. In some embodiments, the detection or labeling reagent is gold nanoparticles (AuNP).

### BRIEF DESCRIPTION OF THE DRAWINGS

Those of skill in the art will understand that the drawings, described below, are for illustrative purposes only. The drawings are not intended to limit the scope of the present disclosure in any way.

FIGS. 1A-1C depict three-dimensional schematics of a representative assay device of the disclosure illustrating component parts. (A) illustrates a representative reader device according to embodiments of the disclosure. (B) illustrates a representative sample collector with assay cassette according to embodiments of the disclosure. (C) illustrates a representative sample collector, including in cross section, according to embodiments of the disclosure.

FIGS. 2A-2B illustrate an embodiment of an immunoassay strip according to embodiments of the disclosure. (A) illustrates specifically a side view of the immunoassay strip showing the different functional areas according to embodiments of the disclosure. (B) illustrates a top view of the immunoassay strip showing the different functional areas along the strip according to embodiments of the disclosure.

FIG. 3 illustrates an alternative embodiment of the immunoassay strip configuration, where the flow is by upward capillary migration within the assay cassette.

FIG. 4 depicts a flow chart and process overview of the assay describing a method of the disclosure for determining analyte concentration in a water sample from sample preparation to quantitative determination and transmission of results via electronic readout through a remote device according to embodiments of the disclosure.

FIGS. 5A-5B show the amino acid sequences of the monoclonal antibody 2C12 light-chain variable region (A) and heavy-chain variable region (B). The complementarity determining regions (CDR) are indicated by bordered regions covering the respective amino acid sequences.

FIGS. 6A-6B illustrate results of lead detection range testing (in ppb) of the immunoassay using nitrocellulose strips according to embodiments of the disclosure. (A) illustrates a Strip 1 that contained 2.5 μg/ml recombinant antibody bound to gold nanoparticles and 40 nM chelator complex according to embodiments of the disclosure. Lanes correspond to: 1) no chelator added; 2) 0 ppb; 3) 0.2 ppb; 4) 0.5 ppb; 5) 1.0 ppb; 6) 1.9 ppb; 7) 3.9 ppb; and 8) 7.8 ppb, respectively. (B) illustrates a Strip 2 that contained 20 μg/ml recombinant antibody bound to gold nanoparticles and 120 nM chelator complex according to embodiments of the disclosure. Lanes correspond to: 1) no chelator added; 2) 0 ppb; 3) 1.0 ppb; 4) 1.9 ppb; 5) 3.9 ppb; 6) 7.8 ppb; 7) 15.5 ppb; and 8) 20.7 ppb, respectively.

FIG. 7 illustrates a schematic of the immunoassay detection system showing labeled monoclonal antibody binding to either test line or control line depending on whether a water sample contains low or high concentrations of analyte (e.g., lead) according to embodiments of the disclosure.

### DETAILED DESCRIPTION OF THE DISCLOSURE

The present disclosure generally relates to a rapid, easy-to-use assay device, compositions and methods of use for the detection of an analyte (e.g., lead) in drinking water or other water sources suspected to contain heavy metal contaminants including lead. The assay can quantitatively detect very low levels of analyte (e.g., total lead at concentrations <1 ppb) in drinking water and other potable water sources while using a minimal number of procedural steps. The assay system is designed to be used by untrained individuals. The present disclosure encompasses diagnostic kits that may contain a diagnostic specific binding assay, and in some embodiments, an immunodiagnostic specific binding assay system. Further, the reader device, due to its simple method steps and accuracy, make it amenable for field use such as in the home, clinic, and point-of-care (POC) settings. The test results are automatically read with limited user interface and provide for a quantitative assessment via electronic (e.g., wireless) communication with a smartphone app.

Unless otherwise noted, terms in the disclosure are to be understood according to conventional usage by those of ordinary skill in the relevant art.

As used herein, the term “sample” encompasses a sample obtained from a potable water source. The sample can be of any potable water source. Such samples include, but are not limited to, tap or drinking water, household plumbing water contained within pipes, bottled water, well water, and filtered water. A sample to be analyzed can be obtained by simple collection and may be from a single source, i.e., not a mixture from different sources. In some embodiments, the sample is a tap or drinking water sample but in no way is limited to tap or drinking water samples.

The lateral or chromatographic flow assay is based on a competitive immunoassay comprising antibodies, chelator complexes, chelator conjugates comprised of chelator and protein, and a detection molecule. In competitive assays, the analyte and labeled detection molecule or reporter groups are simultaneously introduced to the binding agent such that these molecules compete for binding sites. For competitive immunoassays, the label is typically a labeled analyte conjugate or complex that competes with any unlabeled analyte present in the sample for binding to an antibody. In such competitive assays, the analyte and labeled reporter molecule are simultaneously introduced to the binding agent such that these molecules compete for binding sites.

Provided herein is a method of detecting and quantitating an analyte in a sample comprising the steps of: a) obtaining a sample potentially containing an analyte of interest; b) transferring the sample to a sample collector pre-treating the sample to prepare the sample for detection and quantitation of the analyte; c) contacting the pre-treated sample with a test strip; d) assaying the pre-treated sample using the test strip to determine the concentration of the analyte in the pre-treated sample; and e) quantitating the concentration of the analyte, wherein the concentration of the analyte is measured qualitatively and quantitatively using the test strip.

In some embodiments, the heavy metal detection level in the pre-treated sample is at a concentration of between about <1 ppb to about 20 ppb. In some embodiments, the concentration of metal in the pre-treated sample is between <1 ppb and about 15 ppb, between <1 ppb and about 8 ppb, between <1 ppb and 5 ppb, between <1 ppb and 2 ppb. In some embodiments, the lead Pb(II) concentration in the pre-treated sample is <1 ppb.

In embodiments, the method of detecting and quantitating further comprises the step of: f) determining the concentration level of a metal detected in step d) within 60 minutes of transferring the sample in step b). In embodiments, the concentration level is determined within 45 minutes of transferring the sample in step b). In embodiments, concentration level is determined within 30 minutes of transferring the sample in step b). In embodiments, the concentration level is determined within 20 minutes of transferring the sample in step b). In embodiments, the concentration level is determined within 15 minutes of transferring the sample in step b).

In an alternative competitive assay format, the reporter group or detection molecule e.g., AuNP is covalently linked to the antibody and the competition for antibody binding sites is between analyte (e.g., Pb(II)) complexed to a ligand and analyte complexed to a conjugate, i.e., CHXADTPA vs. CHXADTPA-BSA.

The sample collector is a multi-chamber system that enables a user to collect a sample of water from an in-home faucet or other consumable water source for preparation prior to introduction to the lateral flow assay. The system consists of multiple chambers where a precise amount of sample water is (i) acidified, for example with nitric acid, to solubilize lead contained in particulates, (ii) neutralized, (iii) allowed to flow through an ion-exchange resin to trap metals, (iv) washed with an ammonium acetate solution to remove metals common in drinking water that may interfere with the downstream lead immunoassay (e.g., calcium, magnesium), (v) washed with a higher concentration ammonium acetate solution to elute lead from the resin for capture into a concentrated solution, and (vi) combined with metal chelators and buffers prior to precise metering onto the lateral or chromatographic flow assay. The flow assay cassette is integrated with the sample collection device, and may or may not require a user step to insert into the sample collector. Collectively, these two items, the flow assay cassette and the sample collection device, are referred to as a “consumable” and are used together once in the course of performing an individual analyte test.

The opto-electronic reader consists of an optical read-head, PCBA and Bluetooth (BLE) transmitter that interprets and communicates quantitative detection information to a proprietary user application on the consumer's smart phone. Further, the reader contains a motor drive that both actuates the various steps of the consumable from sample preparation through automated movement of the lateral flow assay at the time a test result is ready to allow the read-head to scan the result. In some embodiments, the reader is powered by replaceable batteries and designed to be used about 20 to 100 times, each time with a new consumable as the end-user performs a new test. In some embodiments, the reader is designed to be used about 30 to 90 times, about 40 to 80 times, about 50 to 70 times or about 60 to 65 times.

The disclosure further provides a method of detecting and quantitating an analyte in the sample comprising an immunoassay test strip, wherein a sample application area and a detection area are on a chromatographic immunoassay test strip, and the test strip further comprises a sample pad, wherein the pre-treated sample is applied to or first contacted with the test strip and a conjugate area or pad, wherein the conjugate pad comprises at least one labeled binding partner that is able to migrate with the sample medium, and a capture area or pad, wherein the capture pad comprises a test line comprising chelator-conjugates and a control line comprising anti-species antibodies, and an absorbant pad, wherein the sample flow terminates.

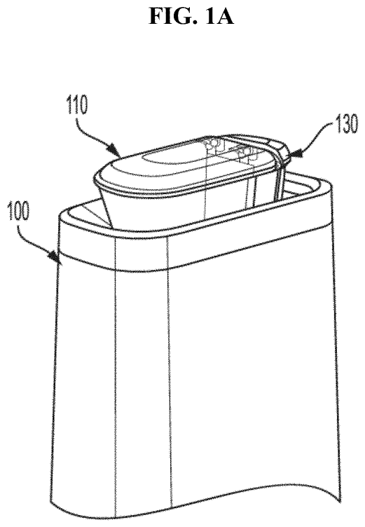

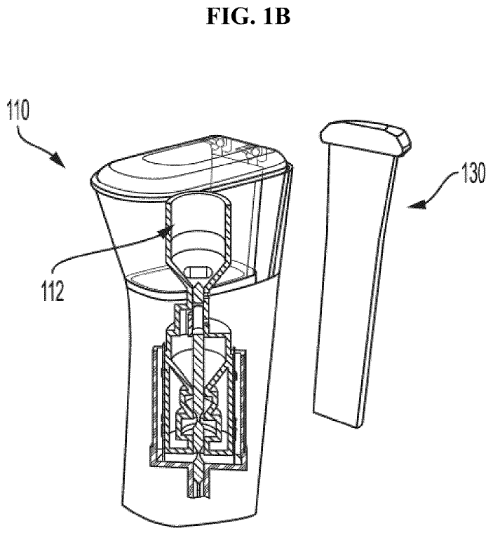

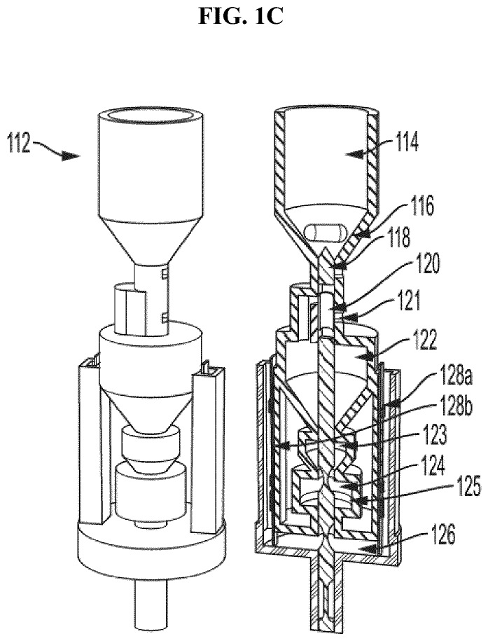

To assist in understanding the present disclosure, FIG. 1 illustrates a representative design and internal working components of the assay detection system according to embodiments of the disclosure. The system is comprised of a reader device, a sample collector and an assay cassette. FIG. 1A illustrates a representative design of the analyte detection system reader device 100 pictured with a consumable sample collector 110 containing an inserted assay cassette 130. FIG. 1B illustrates an embodiment of the sample collector 110 showing a representative sample pretreatment system 112 and a cross sectional detail (right) of an embodiment of the sample pretreatment system 112 and the assay cassette 130. FIG. 1C illustrates an embodiment of the sample pretreatment system 112 contained within the sample collector. As can been seen in the cross-sectional detail of 112, the pretreatment system includes a representative water collection chamber 114, which collects a water sample that contains an analyte (e.g., lead) for testing. A blister or ampoule containing nitric acid 116 is used to lower the sample pH into a desirable range (e.g., <1) to dissolve any analyte that may be in particulate or colloidal form. The pH of the acidified sample is then adjusted to −3.5 by release of acetate buffer and dissolved hydroxide salt (sodium or potassium) from a blister pack (not shown) and then advanced by a distribution rod 118 into an ion-exchange resin column 120, which may or may not be suspended in buffer. Sample then passes into a drain 121 where it flows into a waste collection chamber 122. A second blister pack or equivalent containing ammonium acetate solution and which may or may not be located in chamber 114 is then actuated by the distribution rod 118 and allowed to flow through the resin column 120 whereby the output is collected in another waste chamber adjacent to 122. A third ampoule or blister pack of higher concentration ammonium acetate which may be located in water collection chamber 114 is then actuated by the distribution rod 118 and allowed to flow through the resin column 120 whereby the output is collected in a separation chamber 123 followed by sample splitting chambers 124 and neutralized with a buffer and two different concentrations of chelator(s) contained in blisters or equivalent 125. At the base of the sample collector cassette are located two sample distribution chambers 126, which are each in direct contact with each of the two immunoassay strips 128 with which they are matched. The sample moves therefrom by capillary migration in an upward direction along each of the immunoassay strips 128a and 128b. In some embodiments, the multi-use reader device 100 is comprised of a plastic housing with an internal, motor-driven mechanical design to actuate the fluidics of the sample collector 110 and the assay cassette 130 containing one, two or three immunoassay strips, an opto-electronics assembly for sensing, analyzing, powering and communications, and a software platform consisting of a smartphone “app” and back-end enterprise software system. In some embodiments, once the analyte concentration is determined by the reader after a single use, the consumable sample collector can be disposed of or recycled.

The multi-use reader device allows an untrained user to perform multiple assays successively or at different times. In some embodiments, the multi-use reader device is packaged with, for example, 1-3 single-use consumables each consisting of the assay cassette, and a sample collection and preparation system. The single-use consumable may be disposed of or recycled immediately after the results are analyzed by the reader and transmitted by Bluetooth communication to the smartphone app. In some embodiments, the multi-use reader device provides a quantitative evaluation of an analyte (e.g., total lead) with a high sensitivity, high specificity and wide dynamic range of detection (e.g., <1 to 25 ppb) for any sample of potable or consumable water. In some embodiments, the multi-use reader device comes equipped with Bluetooth connectivity capability for communicating wirelessly to a smartphone with integration to a SafeSpout app. In some embodiments, the multi-use reader device comes equipped with a simple reader operation with an app-driven user interface to easily guide untrained users through the analyte detection process and facile reporting of test results. In some embodiments, the multi-use reader device comes equipped with a robust reader design with storage capability and operating conditions suitable for home, office or field use.

In some embodiments, the multi-use reader device has dimensions of from 50 mm×140 mm×30 mm (W×H×D) and is water resistant. In some embodiments, the multi-use reader device is powered by replaceable double AA batteries.

The multi-use reader devices disclosed herein can be made more versatile by additional design considerations such as by increasing sampling functionality, increasing the number of analyte species for testing, for example, by altering the specificity of the monoclonal antibodies or antibody fragments contained in the test line, etc., increasing Bluetooth capability and electronics functionality, among other design modifications for increasing the versatility and functionality of the system. In some embodiments, the multi-use reader devices have sample collection elements incorporated into the design to facilitate sample handling and treatment prior to commencing the assay. In some embodiments, the multi-use reader devices have sample pre-treatment elements incorporated into the design to adequately prepare the water sample prior to commencing the assay. In some embodiments, the multi-use reader devices are designed to incorporate multiple assays for different analytes. For example, the reader devices can assay for the heavy metal contaminants including, but are not limited, to Pb(II), Cr(II), Ar(II), Cd(II), and Hg(II). In some embodiments, the multi-use reader devices are designed to incorporate increasingly sophisticated Bluetooth microprocessors with accompanying embedded software to transmit more data and communicate more efficiently with SafeSpout's smartphone app. In some embodiments, the multi-use reader devices are designed to incorporate a power management system to regulate the device's power at time of use. In some embodiments, the consumables and all reagent and chemical components are designed to have a 12 to 24-month or longer shelf life. In some embodiments, the multi-use reader devices are designed to be used for 20-100 tests, each using a new consumable. In some embodiments, the multi-use reader devices are designed to be used for about 30-90 tests, about 40-80 tests, about 50-70 tests or about 60-65 tests. In some embodiments, the multi-use reader devices and consumables are comprised of materials that are recyclable.

### Antibodies, Peptides, and Polypeptides

In some embodiments, the method provides a binding partner comprising a monoclonal antibody, a chimeric antibody, a CDR-grafted antibody, a single-chain (scFv) antibody, disulfide-linked Fvs (sdFv), Fab fragments, F(ab′) fragments, or antigen-binding fragments. In some embodiments, the antibody for use in the assay methods described here are monoclonal antibodies (mAb) or recombinant monoclonal antibodies (recmAb), antibody fragments, peptides, or polypeptides that bind to the Pb(II)-CHXADTPA complex and the Pb(II)-CHXADTPA conjugate with different or similar affinities such that upon the binding of the monoclonal antibodies to the Pb(II) are further blocked from or highly diminished in their ability to bind other antigens.

In some embodiments, the monoclonal antibodies or recombinantly synthesized monoclonal antibodies used in the assay for the detection of an analyte such as Pb(II) is termed “2C12” and the generation of this monoclonal antibody or synthetic recombinant monoclonal antibody will be described infra. In some embodiments, the monoclonal antibody is the monoclonal antibody designated 2C12, wherein 2C12 comprises light-chain and heavy-chain variable regions.

In some embodiments, the light-chain and heavy-chain variable regions of monoclonal antibody 2C12 consist of the amino acid sequences of SEQ ID NO: 1 and SEQ ID NO: 2, respectively; wherein the complementarity-determining regions (CDR) in the light chain variable region of 2C12 consist of residues 24-39 (SEQ ID NO: 3), residues 55-61 (SEQ ID NO: 4) and residues 95-103 (SEQ ID NO: 5); and wherein the complementarity-determining regions (CDR) in the heavy-chain variable region of 2C12 consist of residues 26-35 (SEQ ID NO: 6), residues 50-65 (SEQ ID NO: 7) and residues 98-105 (SEQ ID NO: 8).

In some embodiments, the immunoassay is a competitive immunoassay and the assay strip has Pb(II)-CHXADTPA-BSA conjugates immobilized on the test line and an anti-species (e.g., mouse) antibody immobilized on the control line. In some embodiments, a water sample suspected to contain a contaminating analyte such as lead is added to a sample pad for lateral flow or to a sample preparation system for upward flow, where the Pb(II) and the chelator CHXADTPA form a complex, Pb(II)-CHXADTPA. The Pb(II)-CHXADTPA complex flows along the strip by capillary migration and mixes with the antibody on the conjugate pad. The anti-Pb(II)-CHXADTPA antibody, i.e., 2C12, binds to the soluble Pb(II)-CHXADTPA contained in the water sample. In some embodiments, when the concentration of lead is low, most of the anti-Pb(II)-CHXADTPA antibody binds to the test line, providing a strong signal. In some embodiments, when the concentration of lead in the sample is high, and therefore the concentration of Pb(II)-CHXADTPA complex is high, the Pb(II)-CHXADTPA complex competes with the immobilized Pb(II)-CHXADTPA-conjugate at the test line and the intensity of the signal decreases in proportion to the concentration of Pb(II) in the sample.

In some embodiments, the immunoassay of the disclosure comprises at least one assay strip designed to detect a contaminating analyte (e.g., lead) in a range of from <1 ppb (μg/l) to about 25 ppb, or more. In some embodiments, the immunoassay of the disclosure comprises at least two, at least three, or more strips to detect a contaminating analyte (e.g., lead) in a range of from <1 ppb to about 25 ppb, or more.

In some embodiments, the immunoassay of the disclosure comprises two separate assay strips wherein one of the strips detects lead in a range of from 0 to 8 ppb (low range) and the anti-Pb(II)-CHXADTPA antibody is bound to gold (Au) nanoparticles and the Pb(II)-CHXADTPA chelator is present at 40 nM (equivalent to 220 μg/ml), and wherein the second strip detects lead in a range of from 1 to 20 ppb (high range), the anti-Pb(II)-CHXADTPA antibody (e.g., 2C12) is bound to gold (Au) nanoparticles and the Pb(II)-CHXADTPA chelator is present at 120 nM (equivalent to 660 μg/ml).

In some embodiments, the immunoassay of the disclosure comprises three or more assay strips wherein each strip detects the contaminating analyte in a different, but overlapping, range, thus improving the specificity or sensitivity, or both, of the assay. For example, one immunoassay strip may detect 0 to 8 ppb of an analyte, a second immunoassay strip may detect 5 to 15 ppb of an analyte and a third immunoassay strip may detect 10 to 25 ppb of an analyte.

In an alternative embodiment, an antibody, peptide, or polypeptide of the disclosure may bind to the Pb(II)-CHXADTPA complex or Pb(II)-CHXADTPA-conjugate, or both equally and depending on the timing of the formation of the Pb(II)-CHXADTPA complex will thereby compete for the interaction of the monoclonal or recombinant monoclonal antibody binding domains at the test line. In some embodiments, the Pb(II)-CHXADTPA complex is placed in the sample pad and forms immediately after a water sample is applied to the sample port. In some embodiments, free chelator CHXADTPA, is directly mixed with the water sample as part of the sample preparation process before being applied to the immunoassay strip. In some embodiments, the reporter conjugate is embedded in the conjugate pad of the immunoassay strip downstream of the sample port and sample pad such that all or a significant majority of the Pb(II) is bound to the chelator complex, except in those cases where the concentration of the Pb(II) in the sample exceeds the capacity of the chelator complex to bind all the Pb(II) in the water sample. In some embodiments, the gold nanoparticles are passively absorbed onto the monoclonal antibody, but are not covalently bound to the conjugate, to allow color detection at the test and control lines on the immunoassay strip. In some embodiments, the gold nanoparticles are covalently bound to the monoclonal antibody, but are not covalently bound to the conjugate, to allow color detection at the test and control lines on the immunoassay strip.

The antibodies for use in the methods described herein are monoclonal antibodies, including in some embodiments antibodies synthesized from protein expression vectors. Alternatively, the antibodies for use in the methods described herein may be monoclonal antibodies, including in some embodiments mouse antibodies, camelised antibodies, chimeric antibodies, CDR-grafted antibodies, single-chain Fvs (scFv), disulfide-linked Fvs (sdFv), Fab fragments, F(ab′) fragments, or antigen-binding fragments of any of the foregoing. The antigen-binding fragments are fragments of the immunoglobulin molecules that contain a Pb(II)-chelator binding site. Fab, Fab′, F(ab′)₂ and Fv fragments lack the heavy chain constant fragment (Fc) of an intact antibody and may be preferable over an intact antibody. Such fragments are produced from intact antibodies using methods well known in the art, for example by proteolytic cleavage with enzymes such as papain (to produce Fab fragments) or pepsin (to produce F(ab′)₂ fragments). In some embodiments, the antigen-binding fragment is a dimer of heavy chains, a single-chain Fvs (scFv), a disulfide-linked Fvs (sdFv), an Fab fragment, or a F(ab′) fragment. Such fragments may also be fused to another immunoglobulin domain including, but not limited to, an Fc region or fragment thereof. The skilled person will appreciate that other fusion products may be generated, including but not limited to, scFv-Fc fusions, variable region (e.g., VL and VH)-Fc fusions, and scFv-scFv-Fc fusions. Immunoglobulin molecules can be of any type, including, IgG, IgE, IgM, IgD, IgA and IgY, and of any class, including IgG₁, IgG₂, IgG₃, IgG₄, IgA₁ and IgA₂), or of any subclass. In some embodiments, the antibodies used in the methods of the present disclosure are IgG. In some embodiments, the antibodies used are IgG₁.

As noted above, the antibodies for use in the methods described here may be monoclonal antibodies. A monoclonal antibody is derived from a substantially homogeneous population of antibodies specific to a particular antigen, which population contains substantially similar epitope binding sites. Such antibodies may be of any immunoglobulin class including IgG, IgM, IgE, IgA and IgY and any subclass thereof. Methods for the production of recombinant monoclonal antibodies from protein expression vectors are well known in the art. Methods for antibody production from conventional hybridoma technology are also well known in the art. A monoclonal antibody for use in the methods and compositions of the present disclosure may be produced using recombinant technology and is described in the examples infra. In an alternative embodiment, the monoclonal antibody is an antibody generated through conventional hybridoma technology, well known to those with the relevant skill in the art.

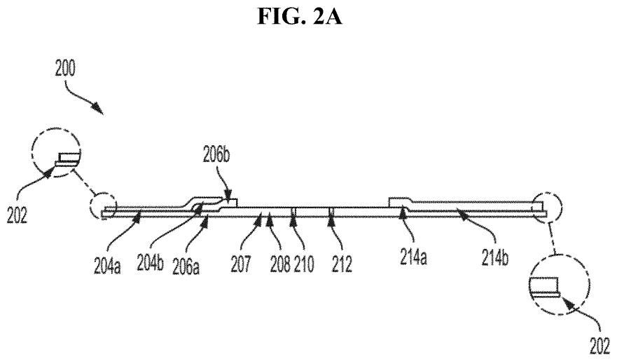

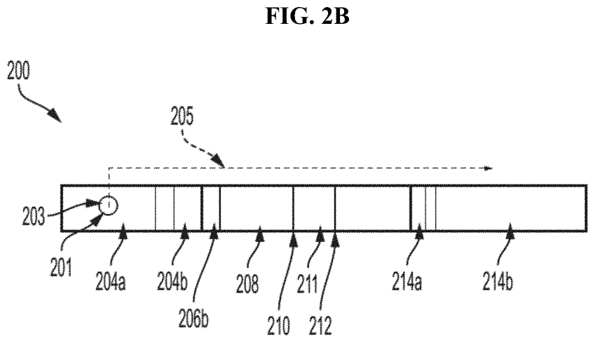

FIG. 2 further assists in illustrating an embodiment of the present disclosure using monoclonal antibodies in an immunoassay. FIG. 2 illustrates a side view of the assay and is generally indicated by 200. Referring to FIG. 2A, the immunoassay strip is comprised of pads or areas where different activities occur as the water sample flows laterally or up through the different pads along the strip. The immunoassay strip lays on top of a thin laminate plate 202 at each end of the assay cassette but thickens in the middle portion of the plate at 207. In some embodiments, a water sample is applied to a sample receiving pad 204a where it migrates to a dried sample treatment pad region 204b which may contain chelator. The analyte (e.g., Pb(II)) in the water sample rapidly complexes with embedded chelator (e.g., CHXADTPA) at the sample treatment pad 204b. In some embodiments, the water sample is first treated with chelator in a pre-treatment step before application to the sample receiving pad, obviating the need for the sample treatment pad 204b. In some embodiments, where the Pb(II) concentration in the sample is suspected to be high, the water sample can first be treated with chelator (e.g., CHXADTPA) applied to the sample receiving pad 204a and allowed to migrate to the sample treatment pad 204b where excess Pb(II) is complexed to form Pb(II)-CHXADTPA. From 204b, the sample flows laterally or upwards to an adjacent conjugate pad 206a which lies immediately below the sample treatment pad 204b complexing any excess Pb(II) that has not been previously complexed with free chelator, CHXADTPA, or through random collisions, dissociates from CHXADTPA and binds to a Pb(II)-conjugate. In some embodiments, CHXADTPA-conjugate complexes any excess Pb(II) or dissociated Pb(II) at a first conjugate pad surface 206a and then at a second conjugate pad surface 206b, where Pb(II)-CHXADTPA-conjugates are formed (e.g., Pb(II)-CHXADTPA-BSA). In some embodiments, the ratio of Pb(II)-CHXADTPA to Pb(II)-CHXADTPA-conjugate is about 100:1, the ratio Pb(II)-CHXADTPA to Pb(II)-CHXADTPA-conjugate is about 90:1, the ratio of Pb(II)-CHXADTPA to Pb(II)-CHXADTPA-conjugate is about 80:1, the ratio of Pb(II)-CHXADTPA to Pb(II)-CHXADTPA-conjugate is about 70:1, the ratio of Pb(II)-CHXADTPA to Pb(II)-CHXADTPA-conjugate is about 60:1, the ratio of Pb(II)-CHXADTPA to Pb(II)-CHXADTPA-conjugate is about 50:1, the ratio of Pb(II)-CHXADTPA to Pb(II)-CHXADTPA-conjugate is about 40:1, the ratio of Pb(II)-CHXADTPA to Pb(II)-CHXADTPA-conjugate is about 30:1, the ratio of Pb(II)-CHXADTPA to Pb(II)-CHXADTPA-conjugate is about 20:1, and the ratio of Pb(II)-CHXADTPA to Pb(II)-CHXADTPA-conjugate is about 10:1. In some embodiments, the bound labeled antibody conjugate with Pb(II)-CHXDTPA migrate from the conjugation pad 206b such that the free CHXADTPA chelator and unbound Pb(II) flows faster laterally or up through capillary migration than the Pb(II)-conjugate into an elongated analyte detection capture pad 208 where it first reaches a test line 210 where the embedded labeled monoclonal antibodies-AuNP in the immunoassay strip bind to the Pb(II)-CHXADTPA-conjugate. The presence and/or amount of an analyte in a sample may be determined by the visibility or lack thereof of a line formed at the test line 210, which is specific for the competition between the labeled antibody with the Pb(II)-CHXADTPA and Pb(II)-CHXADTPA-conjugate, such as a protein. In some embodiments, embedded antibody is passively absorbed onto gold nanoparticles (AuNP). In some embodiments, embedded antibody is covalently linked to gold nanoparticles (AuNP). In some embodiments, Pb(II)-CHXADTPA complexes reach the test line 210 ahead of the Pb(II)-conjugate and limits or significantly reduces the detection signal (e.g., color or fluorescence) at the test line. In some embodiments, the Pb(II)-CHXADTPA reaches the test line 210 and limits or significantly reduces the detection signal (e.g., color or fluorescence) at the test line. In some embodiments, Pb(II)-CHXADTPA-conjugate is already immobilized at the test line. In some embodiments, a low signal at the test line is inversely proportional to the concentration of Pb(II) in the water sample. In some embodiments, a high signal at the test line is inversely proportional to the concentration of Pb(II) in the water sample. In some embodiments, any signal detected at the test line is always inversely proportional to the Pb(II) concentration in the water sample. From there the sample flows to a control line 212, which is used to verify that the reagents are working properly. In some embodiments, the control line 212 is embedded with anti-species specific or anti-protein (e.g., BSA) antibodies covalently linked to gold nanoparticles (AuNP), which target and bind only Pb(II)-CHXADTPA-conjugates or conjugates lacking Pb(II). The sample and any water, buffer, unbound chelator and conjugates flow therefrom through capillary migration through the control line and into an absorbent pad 214a, which acts as a wicking mechanism to regulate capillary flow from the sample receiving pad through to an absorbent reservoir pad 214b.

FIG. 2B illustrates an embodiment of the immunoassay strip of the present disclosure and generally is depicted at 200. Immunoassay strip 200 is shown in expanded top view to illustrate the individual component configuration of the assay elements. Immunoassay strip 200 may be configured to perform at least four types of assays including: secondary antibody sandwich for measurement of sample antibody; antibody-antigen-antibody sandwich for measurement of antigen in either competitive or non-competitive mode; antigen-antibody-antigen sandwich for measurement of antibody; and competitive inhibition assay involving antigen bound to the conjugate nanoparticles, anti-antigen on the strip, with detectable analyte inhibition of the anti-antigen reaction. In some embodiments, the assay configuration is a competitive inhibition assay involving antigen bound to the conjugate nanoparticles, anti-antigen on the strip with a detectable analyte inhibition of the anti-antigen reaction. In some embodiments, the sample receiving pad 204a may contain dried or lyophilized chelator (e.g., CHXADTPA). In an embodiment of the immunoassay device of the present disclosure, the chelator may be added as a step of the sample preparation process. The dried or lyophilized conjugate pad 206a and 206b may contain conjugates comprised of a chelator (metal sols), proteins (e.g., BSA), enzymatic groups (e.g., HRP), fluorescent, latex microparticles, colorimetric particles and the like, including in some embodiments gold colloidal particles. As described in FIG. 2A, the immunoassay strip containing the sample receiving pad 204 the conjugate pad 206 the capture pad 208 and the absorbent pad 214 are attached or laid on top of laminate 202 or a semi-rigid material. In some embodiments, the immunoassay strips in cassette 130 and sample preparation system are integrated with a sample collector 110. Before the assay begins, a pre-treatment step of acidifying the water sample to completely dissolve any particulate analyte (e.g., Pb(II)) is performed. In some embodiments, a water sample is treated with an acid including, but not limited to, nitric, formic, hydrochloric, acetic, sulfuric or citric acid. A water sample 203 is applied at a sample port 201 directly to the sample receiving pad 204a where it flows by capillary migration and mixes with chelator complexes (e.g., CHXADTPA) at sample receiving treatment pad 204b. The sample Pb(II)-chelator complexes flow through capillary migration to the adjacent conjugate pad 206a and 206b where any residual Pb(II) binds with CHXADTPA-conjugates to form, for example, a Pb(II)-CHXADTPA-conjugate (e.g., Pb(II)-CHXADTPA-BSA). In some embodiments, the sample volume is large enough to allow continuous flow through to the capture pad. In some embodiments, the sample receiving pad, conjugate pad, capture pad, test and control line areas and absorbent pad are in continuous fluid contact with each other. In some embodiments, the initial water sample volume can be 10 ml or more. In some embodiments, the initial water sample volume is more than 10 ml and up to 25 ml. In some embodiments, the initial water sample is between 10 and 25 ml. In some embodiments, the initial water sample is 10 ml. In some embodiments, reagents utilized in the sample preparation are included and integrated with the immunoassay system and include, for example, buffers, acidifying agents (e.g., nitric acid), ion-exchange resin, ammonium acetate, chelator and other dried or lyophilized reagents stored in blisters or glass ampoules contained in the sample collector system.

Sample 203 as Pb(II)-CHXADTPA complexes and mobile conjugates as Pb(II)-CHXADTPA-BSA next flow through capillary migration into the elongated capture pad 208. Based on the ratio of Pb(II)-CHXADTPA to Pb(II)-CHXADTPA-BSA-conjugates and the difference in molecular weight, the Pb(II)-CHXADTPA flows laterally 205 ahead of the Pb(II) conjugates during migration through the capture pad 208 in the direction of the absorbent pad 214. Once in the capture pad, the Pb(II)-CHXADTPA complexes either alone or along with Pb(II)-conjugates flow towards the test line 210 where they bind an immobilized capture agent (e.g., an antibody). In some embodiments, only the Pb(II)-CHXADTPA complexes flow along the capture pad towards the test line 210. In some embodiments, the immobilized capture agent is a monoclonal antibody targeted against the Pb(II)-CHXDTPA or the Pb(II)-CHXDTPA moiety of the Pb(II)-CHXDTPA-conjugate. In some embodiments, the immobilized monoclonal antibody embedded at the test line 210 is designated 2C12. In some embodiments, the immobilized monoclonal antibody is an antibody fragment such as an Fab, Fab′, F(ab′)₂ and Fv fragments lacking the heavy chain constant fragment (Fc) of an intact antibody and may be in some cases preferable over an intact antibody embedded at the test line 210. The skilled person will appreciate that other fusion products may be generated, including but not limited to, scFv-Fc fusions, variable region (e.g., VL and VH)-Fc fusions, and scFv-scFv-Fc fusions. Immunoglobulin molecules can be of any type, including, IgG, IgE, IgM, IgD, IgA and IgY, and of any class, including IgG₁, IgG₂, IgG₃, IgG₄, IgA₁ and IgA₂, or of any subclass. The Pb(II)-CHXADTPA can bind to the monoclonal antibodies at the test line before the Pb(II)-conjugates and outcompete the conjugates for binding sites. In some embodiments, the ratio of Pb(II)-CHXADTPA to Pb(II)-CHXADTPA-conjugate is about 100:1, the ratio Pb(II)-CHXADTPA to Pb(II)-CHXADTPA-conjugate is about 90:1, the ratio of Pb(II)-CHXADTPA to Pb(II)-CHXADTPA-conjugate is about 80:1, the ratio of Pb(II)-CHXADTPA to Pb(II)-CHXADTPA-conjugate is about 70:1, the ratio of Pb(II)-CHXADTPA to Pb(II)-CHXADTPA-conjugate is about 60:1, the ratio of Pb(II)-CHXADTPA to Pb(II)-CHXADTPA-conjugate is about 50:1, the ratio of Pb(II)-CHXADTPA to Pb(II)-CHXADTPA-conjugate is about 40:1, the ratio of Pb(II)-CHXADTPA to Pb(II)-CHXADTPA-conjugate is about 30:1, the ratio of Pb(II)-CHXADTPA to Pb(II)-CHXADTPA-conjugate is about 20:1, and the ratio of Pb(II)-CHXADTPA to Pb(II)-CHXADTPA-conjugate is about 10:1. In some embodiments, the Pb(II)-CHXDTPA reaches the test line 210 ahead of the Pb(II)-conjugate and limits or significantly reduces the detection signal (e.g., color or fluorescence) at the test line. In some embodiments, a low signal at the test line is inversely proportional to the concentration of Pb(II) in the water sample. In some embodiments, a high signal at the test line is inversely proportional to the concentration of Pb(II) in the water sample. In some embodiments, any signal detected at the test line is always inversely proportional to the Pb(II) concentration in the water sample.

There is also a control line 212, which is used to verify that the reagents are reacting as they should. In some embodiments, the control line 212 may contain dried or lyophilized, embedded anti-species specific antibodies, which bind only the monoclonal or recombinant reporter labeled conjugate. The dried or lyophilized conjugate may consist of latex microparticles, enzymatic, fluorescent, or visually observable tags such as silver, selenium, carbon, other metal sol tags, including in some embodiments colloidal gold tags to allow detection upon binding of the anti-species specific antibodies. The sample and any water, buffer, unbound chelator and conjugates then flow through capillary migration through the control line area and into the absorbent pad 214a, which acts as a wicking mechanism to regulate the capillary flow from the sample receiving pad through to the absorbent reservoir pad 214b.

The immunoassay strip may be comprised of a series of porous materials such as paper, cotton, polyester, glass, nylon, mixed cellulose esters, spun polyethylene, polysulfones, and the like. In some embodiments, the immunoassay strip is comprised of nitrocellulose, nylon, or mixed cellulose esters are used for the analyte capture pad in the immunoassay strip 200 while paper, cotton, polyester, glass fiber, or polyethylene may be preferred for the conjugate pad 206, sample receiving pad 204 and absorbent pad 214.

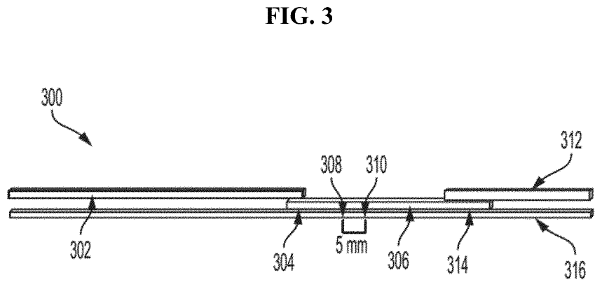

FIG. 3 illustrates an embodiment of the immunoassay strip wherein the chromatographic elements are assembled into an assay platform 300 indicating exemplary component pad sizes including lengths, widths and thicknesses of each element. Exemplary locations and distances of the test and control lines with respect to the chromatographic elements is also shown. Advantageously, as illustrated in FIG. 1C, this embodiment of the assay does not require a sample pad or sample port as the sample moves by gravity to the two sample distribution chambers. Once sample reaches the distribution chambers, it contacts the two immunoassay strips and moves upward by capillary migration up the strip. The sample first contacts the conjugate pad 302, which can have a length of between about 20-30 mm or about 25 mm. The sample flows through the conjugate pad, which overlaps the capture pad 304 by about 2-4 mm, about 2-3 mm or about 2 mm. The capture pad 306 can have a length of between about 20-30 mm or about 25 mm and contains a test line 308 where colored labeled antibodies bind to the Pb(II)-CHXADTPA-conjugate, or minimally to no binding in the presence of Pb; and a control line 310, where anti-species, anti-protein antibodies bind colored labeled antibodies or colored labeled Pb(II)-CHXADTPA-conjugates. An absorbent pad 312 acts as a wick drawing the sample up and can overlap the capture pad 314 by about 5-9 mm, about 6-8 mm or about 6 mm. The entire assay strip is supported by a backing card 316 with a length of between about 50-80 mm, about 60-70 mm or about 60-65 mm. In some embodiments, the length of the backing card is 60 mm.

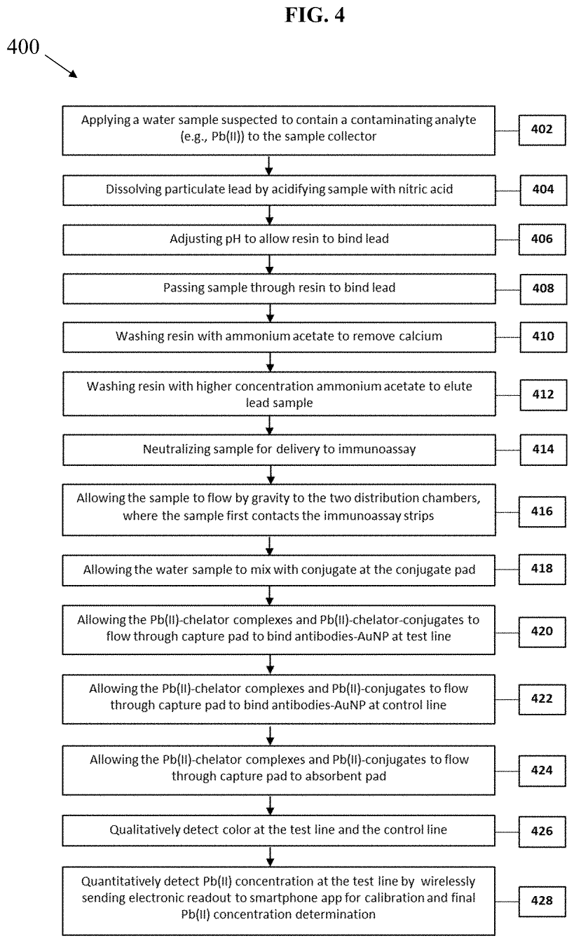

The general process steps including sample preparation of the immunoassay disclosed herein are described in FIG. 4 and in flowchart 400. Steps 402 to 414 constitute pre-treatment steps and are designed to dissolve any particulate heavy metal analyte (e.g., lead), bind the lead to a resin and remove bound calcium for improved specificity of the assay. A water sample suspected to contain lead is captured in the sample collector 402. The sample is acidified (e.g., nitric acid) 404 to dissolve any particulate analyte (e.g., lead). In an alternative embodiment, the water sample is first mixed in a sample pre-treatment step with free chelator to form Pb(II)-chelator complexes before applying the water sample to the sample collector. The pH of the sample is then raised 406 to allow the lead to bind to resin. In some embodiments, the resin is a chelating ion-exchange resin (e.g., Chelex 100; BioRad, Hercules, Calif.). Lead is bound to the resin 408 and the resin is then washed with ammonium acetate to remove calcium 410. After the ammonium acetate wash, the lead is eluted 412 with either ammonium acetate or nitric acid and neutralized 414 before delivery of the sample to the immunoassay 416. The pre-treated sample then flows to the sample distribution chambers at the bottom of the sample collector module, whereupon the sample first contacts the immunoassay strips and the immunoassay begins 416. The metal-containing sample then flows up the immunoassay strips via capillary migration and mixes with labeled antibodies in the conjugate pad 418 where the analyte-chelator complexes bind antibodies. From there, the labeled antibody-Pb(II)-CHXADTPA complexes flow along the capture pad until they reach the test line 420, where they bind Pb(II)-CHXADTPA-conjugates, if the concentration of analyte is low or pass the test line if the analyte concentration is high. In an alternative embodiment, the gold nanoparticles are added to the assay as a separate reagent (e.g., in buffer) after the sample flows by capillary migration from the conjugate pad to the capture pad. The sample, as antibody-Pb(II)-CHXADTPA complexes, then flows through capillary migration to the capture pad until it reaches the test line 420 where Pb(II)-chelator and Pb(II)-conjugates become bound to a partner antibody. In some embodiments, the Pb(II)-chelator moves faster through the capture pad area to bind monoclonal antibody before Pb(II)-conjugates bind monoclonal antibody. In some embodiments, the monoclonal antibodies embedded at the test line are designated 2C12. In some embodiments, the recombinant monoclonal antibodies or non-recombinant monoclonal antibodies embedded at the test line is 2C12 or derivatives, variants or fragments thereof. In some embodiments, the monoclonal antibody is full-length recombinant 2C12 monoclonal antibody embedded at the test line. The capture pad containing detection reagents is selected to have a sufficient pore size such that the conjugate reagent may be comprised of latex, gold, silver, selenium, carbon, but are not limited to these elements. Unbound Pb(II)-CHXADTPA complexes and Pb(II)-CHXDTPA-conjugates at the test line flow through to the capture pad past the test line until they reach the control line 422 where anti-species specific or anti-protein antibodies are embedded. Sample size is determined by the capacity of the absorbent pad 424 and may be in the range of from 50 μl to 150 μl, 60 to 100 μl, or 70 to 80 μl. Excess unbound Pb(II)-CHXADTPA complexes and Pb(II)-CHXDTPA-conjugates then migrate towards the absorbent pad 424. Once the sample liquid has flowed from the control line through to the absorbent pad, the assay is complete and can first be qualitatively assessed 426 with regard to test line and control line color development. Quantitative determination of the analyte concentration 428 is performed by an opto-electronic read-head that scans the test line, interprets the relationship between color intensity and calibrated analyte concentration detected, and transmits via Bluetooth capability the results to a proprietary smartphone app.

### Chemicals and Reagents

The present disclosure describes chemicals and reagents that are required or may be optional depending on the sample used in the assay to achieve robust detection of a target analyte coupled with high specificity.

In some embodiments, the present disclosure provides chelators, buffers, acids, conjugate proteins, detection particles, and reporter groups. In some embodiments, the chelators that may be used in the methods described in the disclosure are selected from the group consisting of CHXADTPA, CHXEDTA, EDTA, EGTA, citrate, and ITCBE or the free acids of any of the foregoing. In some embodiments, the chelator is CHXADTPA or CHXEDTA. In some embodiments, the chelator is CHXADTPA.

In some embodiments, the buffers that can be used in the methods described in the disclosure are any suitable buffers required to maintain the stability of the chelator-conjugate complex to allow the immunoassay to detect an analyte (e.g., lead) in the low ppb range (<1 ppb). In some embodiments, the buffer is selected from the group consisting of HEPES, HEPES-buffered saline (HBS), CHAPS and phosphate buffers. In some embodiments, the buffer is HEPES-buffered saline (HBS).

In some embodiments, the conjugate proteins that may be used in the methods described in the disclosure are selected from the group consisting of bovine serum albumin (BSA) and keyhole limpet hemocyanin (KLH). In some embodiments, the conjugate protein is BSA.

In some embodiments, the reporter groups and detection particles that may be used in the methods described in the disclosure are selected from the group consisting of gold nanoparticles (AuNP), latex microparticles, enzymatic reporter groups such as horseradish peroxidase (HRP) and alkaline phosphatase, metal sol tags such as silver, selenium, and carbon tags. In some embodiments, the reporter groups or detection particles are gold nanoparticles (AuNP).

In some embodiments, the acids that may be used in the methods described in the disclosure are selected from the group consisting of nitric, acetic, hydrochloric, formic, citric, and sulfuric. In some embodiments, the acid is nitric acid.

The present disclosure further provides a system for detecting and quantitating an analyte in a sample comprising the steps of: a) obtaining a sample potentially containing an analyte of interest; b) transferring the sample to a sample collector for pre-treatment to prepare the sample for detection and quantitation of the analyte; c) contacting the pre-treated sample with a test strip; d) assaying the pre-treated sample using the test strip to determine the concentration of the analyte in the sample, wherein the concentration of the analyte is measured qualitatively and quantitatively using the test strip; e) qualitatively determining the concentration level of a metal detected in step d), wherein the result is determined within 60 minutes, within 45 minutes, within 30 minutes, within 20 minutes or within 15 minutes; and f) quantitatively determining the concentration level of a metal detected in step d), wherein the metal concentration level is recorded in a multi-use reader device. In some embodiments, the concentration data is wirelessly transferred to a smartphone app using a back-end software platform. In some embodiments, the sample collector in step b) and parts of the multi-use reader device in step f) are disposable or recyclable.

The present disclosure further provides a device for detecting and quantitating an analyte in a liquid sample comprising: an analyte detection reader 100; a sample collector 110; and an assay cassette 130, wherein the analyte detection reader is designed to hold the sample collector and the assay cassette for reading a sample. In embodiments, the sample collector further comprises a sample pre-treatment system 112. In some embodiments, the sample pre-treatment system 112 comprises: a water collection chamber 114; a blister ampoule containing acid 116; a distribution rod 118; an ion-exchange resin 120; a drain 121; a waste collection chamber 122; a separation chamber 123; sample splitting chambers 124; a blister or equivalent containing neutralizing reagent and chelator(s) 125; sample distribution chambers 126, and immunoassay strips 128a and 128b, wherein the immunoassay strips detect different concentration ranges of analyte.

In some embodiments, the device comprises immunoassay strips for detecting and quantitating an analyte comprising: a base plate 202; a sample receiving pad 204a and a dried sample treatment pad 204b; a first conjugate pad area 206a and a second conjugate pad area 206b, wherein the first conjugate pad area and the second conjugate pad area are impregnated with a first binding partner bound to a detection reagent; and wherein the first binding partner bound to the detection reagent is able to flow along the immunoassay strips to an elongated analyte detection capture pad 208; a first capture area comprising a test line 210 immobilized with analyte chelator-conjugates, wherein the first binding partner bound to a detection reagent competes for binding sites at the test line; a second capture area comprising a control line 212 immobilized with a second binding partner; and an absorbent pad 214a and an absorbent reservoir pad 214b, wherein the absorbent pad acts as a wicking mechanism to regulate capillary flow from the sample receiving pad to the absorbent reservoir pad.

In some embodiments, the device comprises a binding partner comprising a monoclonal antibody, a chimeric antibody, a CDR-grafted antibody, a single-chain (scFv) antibody, disulfide-linked Fvs (sdFv), Fab fragments, F(ab′) fragments, or antigen-binding fragments. In some embodiments, the monoclonal antibody is the monoclonal antibody designated 2C12, wherein 2C12 comprises light-chain and heavy-chain variable regions. In some embodiments, the light-chain and heavy-chain variable regions consist of the amino acid sequences of SEQ ID NO: 1 and SEQ ID NO: 2, respectively; wherein the complementarity-determining regions (CDR) in the light chain variable region of 2C12 consist of residues 24-39 (SEQ ID NO: 3), residues 55-61 (SEQ ID NO: 4) and residues 95-103 (SEQ ID NO: 5); and wherein the complementarity-determining regions (CDR) in the heavy-chain variable region of 2C12 consist of residues 26-35 (SEQ ID NO: 6), residues 50-65 (SEQ ID NO: 7) and residues 98-105 (SEQ ID NO: 8).

In some embodiments, the device further comprises chelators. In some embodiments, the chelators are selected from the group consisting of CHXDTPA, CHXEDTA, EDTA, EGTA, citrate, and ITCBE or the free acids of any of the foregoing. In some embodiments, the chelator is CHXDTPA.

In some embodiments, the device further comprises detection or labeling reagents. In some embodiments, the detection or labeling reagents are selected from the group consisting of gold nanoparticles (AuNP), latex microparticles, reporter groups including horseradish peroxidase (HRP) and alkaline phosphatase (AP), metal sol tags including silver, selenium and carbon. In some embodiments, the detection or labeling reagent is gold nanoparticles (AuNP)

The following examples are provided to illustrate some embodiments of the present disclosure. These examples should not be construed in any way to limit the disclosure to the particular devices, compositions and methods describe.

### EXAMPLES

### Example 1: Preparation of Pb(II)-Conjugates

Protein-chelator conjugates were prepared by a modification of the previously described method of Breshbiel et al. (1986) Inorg. Chem. 25:2772-2781 in a final volume of 500 μL which contained 5 mg of protein (BSA or KLH), 2.6 mM CHX-A, 2.9 mM Pb(NO3)₂, and 46 mM triethyl-amine in 50 mM Hepes buffer, pH 9.0. The pH of the reaction mixture was maintained at 9.0 by the addition of KOH. A metal-free BSA conjugate was prepared by omitting the Pb(NO3)₂ from the reaction mixture. The reactions were stirred at 25° C. for 3 h, and any unreacted low molecular-weight components were removed by buffer exchange using a Centricon-30 filter. The protein conjugates were characterized as described previously by Chakrabarti et al. (1994), Anal. Biochem. 217:70-75. The extent of substitution of free lysine groups was 17.1% for the KLH conjugate and 5.5% for the BSA conjugates.

### Example 2: Generation of Recombinant Monoclonal Antibody Specific to Pb(II)-CHXADTPA Complexes and Conjugates

Recombinant monoclonal antibody, designated 2C12, was generated using the AbAb Recombinant Platform (Absolute Antibody NA, Boston, Mass.) from the sequence determined for the antigenic Pb(II)-CHXDTPA-conjugate mAB 2C12 as described in Khosraviani et al. (2000), Bioconjugate 11:267-277. Briefly, in the first phase of recombinant antibody gene cloning and expression, the 2C12 antibody genes were codon optimized for expression in mammalian cells using the HEK293 cell line prior to scale up synthesis. After optimized expression was determined in HEK293 cells, the sequences were subcloned into an appropriate cloning vector furnished by Absolute Antibody. The second phase consisted of scale up pilot expression and purification. HEK293 cells were passaged at an optimum growth stage for transient transfection. Cells were transiently transfected into an appropriate expression vector and cultured for a further 6-14 days. An appropriate volume of cells was transfected with the aim of obtaining a specified amount (in milligrams) of protein after purification. Cultures were harvested in a one-step purification process using affinity chromatography. After purification, the purified antibodies were exchanged into buffer for long-term storage. The purified 2C12 antibody was analyzed for purity by SDS-PAGE and the concentration was determined by UV spectroscopy. The antibody isotype is IgG₁ and has a molecular weight of 144.7 kDa. The extinction coefficient was determined to be 222,110 M⁻¹ cm⁻¹.

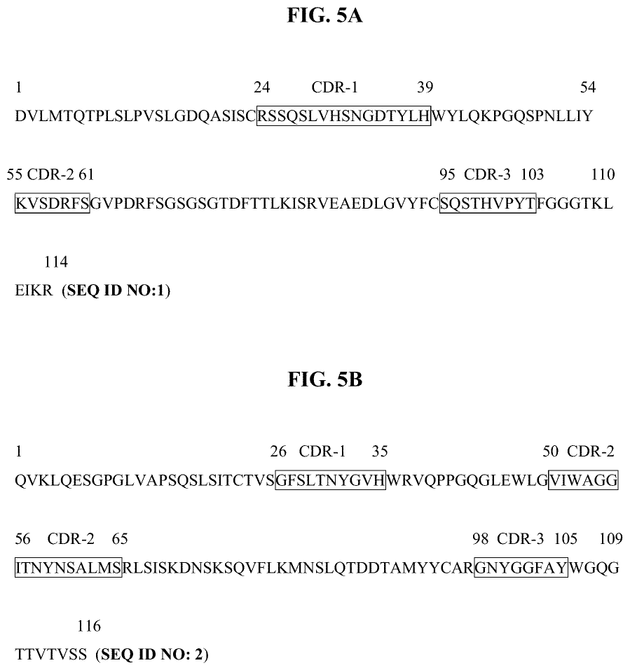

The specific Pb(II)-CHXDTPA complex binding regions of monoclonal antibody 2C12 have been elucidated by Khosraviani et al. (1994). FIGS. 5A-B show the amino acid sequences of the light-chain variable (A) (SEQ ID NO: 1) and heavy-chain variable (B) (SEQ ID NO: 2) regions of 2C12. The complimentarity determining regions (CDR) are indicated as bordered amino acid sequences for the three CDRs contained in each of the two variable regions. The six total CDR contained in the light-chain and heavy-chain variable regions are shown in Table 1.

TABLE 1
Amino acid sequences of the light-chain and heavy-chain variable
regions of monoclonal antibody 2C12.

| ID NO: | 2C12\hat{ } | Sequence* |
|---|---|---|
| SEQ ID NO: 3 | Light CDR-1/24-39 | RSSQSLVHSNGDTYLH |
| SEQ ID NO: 4 | Light CDR-2/55-61 | KVSDRFS |
| SEQ ID NO: 5 | Light CDR-3/95-103 | SQSTHVPYT |
| SEQ ID NO: 6 | Heavy CDR-1/26-35 | GFSLTNYGVH |
| SEQ ID NO: 7 | Heavy CDR-2/50-65 | VIWAGGITNYNSALMS |
| SEQ ID NO: 8 | Heavy CDR-3/98-105 | GNYGGFAY |

*With permission of D. Blake; \hat{ } indicates amino acid residue numbers in 2C12 in Khosraviani et al. 2000.

### Example 3: Water Sample Preparation

Water samples spiked with or suspected of containing lead were subjected to a pre-treatment step to dissolve any particulate lead before being delivered to the immunoassay detection system. Ten milliliters of sample were acidified by the addition of 1.5% nitric acid and left at room temperature for 3.5 minutes to dissolve particulates. The sample pH was adjusted with acetate buffer and sodium hydroxide to achieve a pH of 3.5 for the resin binding step. The sample was added to a chelex resin column to bind the lead and the flow rate (gravity) was adjusted to about 0.5-1 ml/min based on the resin column packing density. Next, the resin was washed with 25 ml of 0.1 M ammonium acetate buffer (pH 3.5) to remove calcium. The lead was selectively eluted from the chelex resin with 15 mL of 1 or 2 M ammonium acetate buffer (pH 4.5). Lastly, the sample containing lead was neutralized with either 1M potassium carbonate or 5M KOH or KOH in the presence of chelator (CHXADTPA) in preparation for immunoassay detection and lead concentration determination.

### Example 4: Determination of Time Required for Particulate Lead Dissolution by Acidification

Water samples known to contain particulate lead (˜30 ppb) were either filtered with a 10 μm syringe filter to remove large particles (sample 1) or left unfiltered (sample 2) and then both samples were split into two replicates (samples 1a, 1b and 2a, 2b) and acidified with 1.5% nitric acid to dissolve the particulate lead into lead 2⁺ ions. Aliquots were removed at 3, 10, 30, 60, 90, 120, 270, and 960 minutes and the concentration of dissolved lead was measured by inductively coupled plasma mass spectrometry using a NexION 350D mass spectrometer connected to a PFA-ST nebulizer and a peltier controlled quartz cyclonic spray chamber set at 4° C. No significant difference in the concentration of ionic lead was observed after three minutes of incubation with 1.5% nitric acid, indicating that three minutes incubation time at room temperature is sufficient to dissolve lead particulates present in the water samples to a repeatable concentration of lead 2⁺ ions (Table 2).

TABLE 2
ICP-MS measurements of lead concentration in filtered and
unfiltered water samples contaminated with lead particulates.
Samples were incubated on 1.5% nitric acid for 3 to 960 minutes.

| Incubation | Sample 1a | Sample 1b | Sample 2a | Sample 2b |
|---|---|---|---|---|
| time (min) | Pb²⁺ (ppb) | Pb²⁺ (ppb) | Pb²⁺ (ppb) | Pb²⁺ (ppb) |
| 3 | 30.3 | 32.3 | 32.2 | 33.4 |
| 10 | 30.2 | 32.9 | 32.2 | 32.6 |
| 30 | 30.6 | 32.9 | 32.6 | 33.1 |
| 60 | 30.4 | 32.3 | 32.7 | 32.7 |
| 90 | 30.9 | 32.1 | 33.0 | 33.3 |
| 120 | 30.2 | 32.3 | 31.9 | 31.9 |
| 270 | 30.5 | 30.4 | 32.4 | 32.6 |
| 960 | 31.1 | 30.9 | 32.8 | 33.9 |

### Example 5: Determining the Pb(II) Detection Range Using Gold Nanoparticles in a Full Strip Format

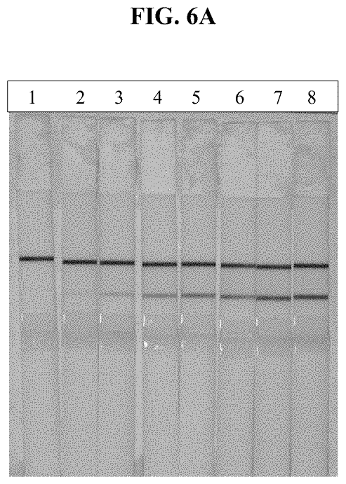

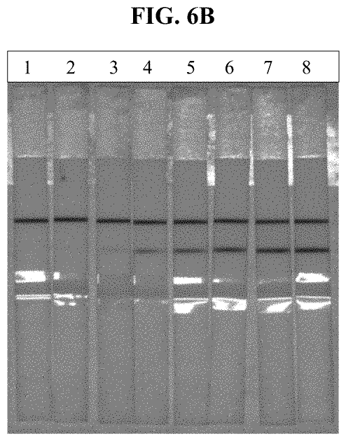

Range testing was performed with 2.5 μg/ml (strip 1) and 20 μg/ml (strip 2) gold conjugate labeled recombinant antibody strips with covertape in a full-strip format. The replicate testing consisted of dried strips with 2.5 μg/ml and 20 μg/ml of recombinant monoclonal 2C12 antibody-gold nanoparticles paired with 1.5 mg/ml Pb(II)-CHXADTPA-BSA on CN95 membrane. The strips were evaluated in dried format with 25 mm conjugate pad, 25 mm nitrocellulose membrane, 18 mm absorbant pad and 8 mm covertape. There was a 2 mm overlap of the conjugate pad onto the nitrocellulose, a 3 mm overlap of covertape onto the nitrocellulose and a 6 mm overlap of the absorbent pad onto the nitrocellulose. In testing the assay, reagents and materials consisted of a 4.8 mM Pb standard (1000 ppm), 10 μM CHXADTPA in HBS, 0.01N and 0.1N HCL solutions, Hepes buffered saline solutions with and without 5% Tween 20, CN95 membrane impregnated with Pb(II)-CHXADTPA-BSA at 1.5 mg/ml and goat anti-MS antibodies at 0.5 mg/ml. Recombinant antibody-gold nanoparticles at 2.5 and 20 μg/ml were dried at OD 10 onto fiberglass materials at 25 mm, then were placed on the strip. Briefly, the method consisted of preparing 1 mM solution of CHXADTPA by adding 5.5 mg of the CHXADTPA chelator into 10 ml of HEPES-buffered saline (HBS). A 1 mM solution of lead, as Pb(II), was initially prepared in HCL to completely dissolve any particulate lead and then further diluted to a working concentrations between 0-2000 nM. For the test procedure, 40 nM and 120 nM of chelator conjugate (CHXADTPA-BSA) was paired with 2.5 μg/ml and 20 μg/ml recombinant 2C12-gold nanoparticles with or without lead, respectively. FIG. 6 shows the results for strip 1 (FIG. 6A) using 40 nM chelator-conjugate at 2.5 μg/ml rec-Ab-Au nanoparticles and strip 2 (FIG. 6B) using 120 nM chelator-conjugate at 20 μg/ml rec-Ab-Au nanoparticles. Sensitivity was slightly reduced in the dried system with full Strip 1, detecting Pb(II) at 0.5 ppb, whereas in the half-Strip format lead was detected at 0.25 ppb. Line morphology was observed in Strip 2, which displayed slightly higher CV. Aggregation was observed in Strip 1 at the covertape-nitrocellulose interface, which once removed cleared through the membrane. The overall functional detection range of Strip 1 was between 0-8 ppb and for Strip 2 was between 1-20 ppb and thus covered the full range of desired detection without any overlaps.

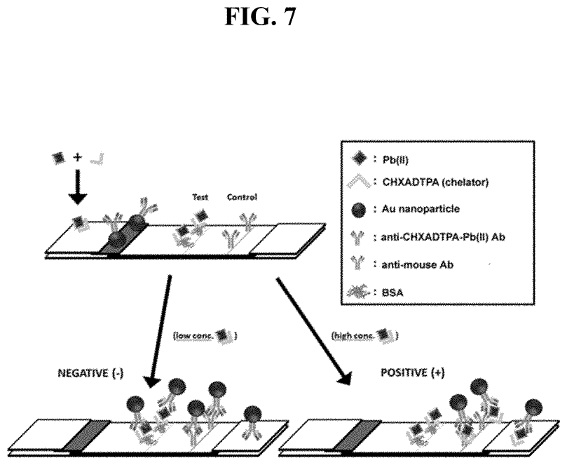

A schematic illustrating Pb(II)-CHXADTPA complexes and Pb(II)-CHXADTPA-conjugates bound to antibody-AuNP at high and low analyte concentrations along an assay strip is shown in FIG. 7.

### Example 6: Kits for the Detection and Quantitation of an Analyte in Water Samples

Assay systems according to the embodiments of the disclosure can be provided in the form of test kits. Such test kits may include one or more single-use consumables (which may be for the same or different analytes), and instructions for the use of the consumables(s) together with the multi-use reader and the Spout app. The instructions will provide direction on how to collect a water sample, integrate the assay cassette with the sample collector, place the integrated consumable into the reader, link the quantitative result of analyte concentration for transmission to the user's smart phone application, and interpret the result relative to different benchmarks. Such instructions may also include standards, such as standard tables, graphs, or pictures for comparison of the results of a test. The analyte detection test kits are envisioned as containing components which will include the reader and one single-use consumable system comprised of a sample collector and preparation materials (e.g., reagents, etc.), and immunoassay strips within a cassette. It is also envisioned that additional single-use consumable systems will be delivered more or less every 6 months, depending on the consumer's choice, in single packages to the consumer's home. Consumers will subscribe to consumable refill quantities from 1 to any number based on their preferences. The reader will be designed to handle approximately 50 or more individual analyte detection tests.

### EQUIVALENTS

Those skilled in the art will recognize or be able to ascertain using no more than routine experimentation, many equivalents to the specific embodiments of the subject matter described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The subject matter is not to be limited in scope by the specific embodiments described herein. Indeed, various modifications of the subject matter disclosed herein in addition to those described herein will become apparent to those skilled in the art from the foregoing description and accompanying figures. Such modifications are intended to fall within the scope of the appended claims.

What is claimed is:

1. A method of detecting and quantitating an analyte in a sample comprising the steps of:
a) obtaining a sample potentially containing an analyte of interest;
b) transferring the sample to a sample collector pre-treating the sample to prepare the sample for detection and quantitation of the analyte;
c) contacting the pre-treated sample with a test strip;
d) assaying the pre-treated sample using the test strip to determine the concentration of the analyte in the pre-treated sample; and
e) quantitating the concentration of the analyte, wherein the concentration of the analyte is measured qualitatively and quantitatively using the test strip.

2. The method of claim 1, wherein the detecting and quantitating of the analyte in the sample comprises an immunoassay test strip, wherein a sample application area and a detection area are on a chromatographic immunoassay test strip, and the test strip further comprises a sample pad, wherein the pre-treated sample is applied to or first contacted with the test strip and a conjugate area or pad, wherein the conjugate pad comprises at least one labeled binding partner that is able to migrate with the sample medium, and a capture area or pad, wherein the capture pad comprises a test line comprising chelator-conjugates and a control line comprising anti-species antibodies, and an absorbant pad, wherein the sample flow terminates.

3. The method of claim 1, wherein the sample is a liquid sample selected from the group consisting of tap water, well water, unfiltered drinking water, filtered drinking water, household plumbing water contained within pipes, bottled water, municipal water, aquifer water, wastewater including industrial wastewater sources, effluent and river water.

4. The method of claim 1, wherein the analyte comprises a heavy metal.

5. The method of claim 4, wherein the heavy metal is selected from lead Pb(II), chromium Cr(II), arsenic Ar(II), cadmium Cd(II), and mercury Hg(II).

6. The method of claim 5, wherein the heavy metal is lead Pb(II).

7. The method of claim 5, wherein the heavy metal detection level in the pre-treated sample is at a concentration of between about <1 ppb to about 20 ppb.

8. The method of claim 7, wherein the concentration of metal in the pre-treated sample is between <1 ppb and about 15 ppb, between <1 ppb and about 8 ppb, between <1 ppb and 5 ppb, between <1 ppb and 2 ppb.

9. The method of claim 7, wherein the lead Pb(II) concentration in the pre-treated sample is <1 ppb.

10. The method of claim 1, further comprising the step of: f) determining the concentration level of a metal detected in step d) within 60 minutes of transferring the sample in step b).

11. The method of claim 10, wherein the concentration level is determined within 45 minutes of transferring the sample in step b).

12. The method of claim 10, wherein the concentration level is determined within 30 minutes of transferring the sample in step b).

13. The method of claim 10, wherein the concentration level is determined within 15 minutes of transferring the sample in step b).

14. The method of claim 2, wherein the at least one binding partner comprises a monoclonal antibody, a chimeric antibody, a CDR-grafted antibody, a single-chain (scFv) antibody, disulfide-linked Fvs (sdFv), Fab fragments, F(ab′) fragments, or antigen-binding fragments.

15. The method of claim 14, wherein the monoclonal antibody is the monoclonal antibody designated 2C12, wherein 2C12 comprises light-chain and heavy-chain variable regions.

16. The method of claim 15, wherein the light-chain and heavy-chain variable regions consist of the amino acid sequences of SEQ ID NO: 1 and SEQ ID NO: 2, respectively; wherein the complementarity-determining regions (CDR) in the light chain variable region of 2C12 consist of residues 24-39 (SEQ ID NO: 3), residues 55-61 (SEQ ID NO: 4) and residues 95-103 (SEQ ID NO: 5); and wherein the complementarity-determining regions (CDR) in the heavy-chain variable region of 2C12 consist of residues 26-35 (SEQ ID NO: 6), residues 50-65 (SEQ ID NO: 7) and residues 98-105 (SEQ ID NO: 8).

17. The method of claim 2, further comprising chelators.

18. The method of claim 17, wherein the chelators are selected from the group consisting of CHXDTPA, CHXEDTA, EDTA, EGTA, citrate, and ITCBE or the free acids of any of the foregoing.

19. The method of claim 18, wherein the chelator is CHXDTPA.

20. The method of claim 1, further comprising detection or labeling reagents.

21. The method of claim 20, wherein the detection or labeling reagents are selected from the group consisting of gold nanoparticles (AuNP), latex microparticles, reporter groups including horseradish peroxidase (HRP) and alkaline phosphatase (AP), metal sol tags including silver, selenium and carbon.

22. The method of claim 21, wherein the detection or labeling reagent is gold nanoparticles (AuNP).

23. A system for detecting and quantitating an analyte in a sample comprising the steps of:
a) obtaining a sample potentially containing an analyte of interest;
b) transferring the sample to a sample collector for pre-treatment to prepare the sample for detection and quantitation of the analyte;
c) contacting the pre-treated sample with a test strip;
d) assaying the pre-treated sample using the test strip to determine the concentration of the analyte in the sample, wherein the concentration of the analyte is measured qualitatively and quantitatively using the test strip;
e) qualitatively determining the concentration level of a metal detected in step d), wherein the result is determined within 60 minutes, within 45 minutes, within 30 minutes, within 20 minutes or within 15 minutes; and
f) quantitatively determining the concentration level of a metal detected in step d), wherein the metal concentration level is recorded in a multi-use reader device.

24. The system of claim 23, wherein the concentration data is wirelessly transferred to a smartphone app using a back-end software platform.

25. The system of claim 23, wherein the sample collector in step b) and parts of the multi-use reader device in step f) are disposable or recyclable.

26. A device for detecting and quantitating an analyte in a liquid sample comprising:
an analyte detection reader 100;
a sample collector 110; and
an assay cassette 130, wherein the analyte detection reader is designed to hold the sample collector and the assay cassette for reading a sample.

27. The device of claim 26, wherein the sample collector further comprises a sample pre-treatment system 112.

28. The device of claim 27, wherein the sample pre-treatment system 112 comprises:
a water collection chamber 114,
a blister ampoule containing acid 116,
a distribution rod 118,
an ion-exchange resin 120,
a drain 121,
a waste collection chamber 122,
a separation chamber 123,
sample splitting chambers 124,
a blister or equivalent containing neutralizing reagent and chelator(s) 125, sample distribution chambers 126, and
immunoassay strips 128a and 128b, wherein the immunoassay strips detect different concentration ranges of analyte.

29. The device of claim 28, wherein the immunoassay strips are for detecting and quantitating an analyte comprising:
a base plate 202;
a sample receiving pad 204a and a dried sample treatment pad 204b;
a first conjugate pad area 206a and a second conjugate pad area 206b, wherein the first conjugate pad area and the second conjugate pad area are impregnated with a first binding partner bound to a detection reagent; and wherein the first binding partner bound to the detection reagent is able to flow along the immunoassay strips to an elongated analyte detection capture pad 208;
a first capture area comprising a test line 210 immobilized with analyte chelator-conjugates, wherein the first binding partner bound to a detection reagent competes for binding sites at the test line;
a second capture area comprising a control line 212 immobilized with a second binding partner; and
an absorbent pad 214a and an absorbent reservoir pad 214b, wherein the absorbent pad acts as a wicking mechanism to regulate capillary flow from the sample receiving pad to the absorbent reservoir pad.

30. The device of claim 29, wherein the first binding partner comprises a monoclonal antibody, a chimeric antibody, a CDR-grafted antibody, a single-chain (scFv) antibody, disulfide-linked Fvs (sdFv), Fab fragments, F(ab′) fragments, or antigen-binding fragments.

31. The device of claim 30, wherein the monoclonal antibody is the monoclonal antibody designated 2C12, wherein 2C12 comprises light-chain and heavy-chain variable regions.

32. The device of claim 31, wherein the light-chain and heavy-chain variable regions consist of the amino acid sequences of SEQ ID NO: 1 and SEQ ID NO: 2, respectively; wherein the complementarity-determining regions (CDR) in the light chain variable region of 2C12 consist of residues 24-39 (SEQ ID NO: 3), residues 55-61 (SEQ ID NO: 4) and residues 95-103 (SEQ ID NO: 5); and wherein the complementarity-determining regions (CDR) in the heavy-chain variable region of 2C12 consist of residues 26-35 (SEQ ID NO: 6), residues 50-65 (SEQ ID NO: 7) and residues 98-105 (SEQ ID NO: 8).

33. The device of claim 29, wherein the second binding partner comprises an anti-species antibody.

34. The device of claim 29, further comprising chelators.

35. The device of claim 34, wherein the chelators are selected from the group consisting of CHXDTPA, CHXEDTA, EDTA, EGTA, citrate, and ITCBE or the free acids of any of the foregoing.

36. The device of claim 35, wherein the chelator is CHXDTPA.

37. The device of claim 29, further comprising detection or labeling reagents.

38. The device of claim 37, wherein the detection or labeling reagents are selected from the group consisting of gold nanoparticles (AuNP), latex microparticles, reporter groups including horseradish peroxidase (HRP) and alkaline phosphatase (AP), metal sol tags including silver, selenium and carbon.

39. The device of claim 38, wherein the detection or labeling reagent is gold nanoparticles (AuNP).


SEQUENCE LISTING (8 sequences)

SEQ ID NO: 1 - protein, 114 aa, Artificial Sequence
  POLYPEPTIDE (1)..(114): Amino acid sequence of 2C12 light chain
DVLMTQTPLSLPVSLGDQASISCRSSQSLVHSNGDTYLHWYLQKPGQSPNLLIYKVSDRFSGVPDRFSGSGSGTDFTTLKISRVEAEDLGVYFCSQSTHVPYTFGGGTKLEIKR

SEQ ID NO: 2 - protein, 116 aa, Artificial Sequence
  POLYPEPTIDE (1)..(116): Amino acid sequence of 2C12 heavy chain
QVKLQESGPGLVAPSQSLSITCTVSGFSLTNYGVHWRVQPPGQGLEWLGVIWAGGITNYNSALMSRLSISKDNSKSQVFLKMNSLQTDDTAMYYCARGNYGGFAYWGQGTTVTVSS

SEQ ID NO: 3 - protein, 16 aa, Artificial Sequence
  PEPTIDE (1)..(16): Light Chain CDR-1
RSSQSLVHSNGDTYLH

SEQ ID NO: 4 - protein, 7 aa, Artificial Sequence
  PEPTIDE (1)..(7): Light Chain CDR-2
KVSDRFS

SEQ ID NO: 5 - protein, 9 aa, Artificial Sequence
  PEPTIDE (1)..(9): Light Chain CDR-3
SQSTHVPYT

SEQ ID NO: 6 - protein, 10 aa, Artificial Sequence
  PEPTIDE (1)..(10): Heavy Chain CDR-1
GFSLTNYGVH

SEQ ID NO: 7 - protein, 16 aa, Artificial Sequence
  PEPTIDE (1)..(16): Heavy Chain CDR-2
VIWAGGITNYNSALMS

SEQ ID NO: 8 - protein, 8 aa, Artificial Sequence
  PEPTIDE (1)..(8): Heavy Chain CDR-3
GNYGGFAY